(19) United States
(12) Patent Application Publication
(10) Pub. No.: US 2026/0338695 A1
(43) Pub. Date: Oct. 29, 2026

(54) A HAND-HELD PROBE FOR A RAMEN SPECTROSCOPY SYSTEM AND A RAMEN SPECTROSCOPY SYSTEM

(71) Applicant: Agency for Science, Technology and Research, Singapore (SG)

(72) Inventors: Malini Olivo, Singapore (SG); Renzhe Bi, Singapore (SG); Ruochong Zhang, Singapore (SG); Yi Qi, Singapore (SG); Poongkulali Rajarahm, Singapore (SG)

(21) Appl. No.: 19/104,955

(22) PCT Filed: Aug. 29, 2023
(86) PCT No.: PCT/SG2023/050589

§ 371 Date: Feb. 19, 2025

(30) Foreign Application Priority Data
Sep. 6, 2022 (SG) 10202250917Y

(51) Int. Cl. G01N 21/65

(52) U.S. Cl. CPC G01N 21/65; G01N 2201/0221; G01N 2201/0633; G01N 2201/0636

(57) ABSTRACT

In some aspects, a hand-held probe for a Raman spectroscopy system is provided. The hand-held probe includes an excitation module, configured to emit a light beam along a light path; a beam splitter, oriented to reflect the light beam along the observation axis; an objective module, configured to receive the reflected light beam and transmit the reflected light beam to an object so as to generate a scattered light beam by the object, and configured to receive and transmit the scattered light beam to the beam splitter; a focusing mirror, configured to focus the refracted light beam from the beam splitter to a focus so as to generate a focused light beam; and an emission module, disposed at the focus and configured to receive the focused light beam, wherein the emission module is disposed at a same side as the excitation module with respect to the observation axis.

### TECHNICAL FIELD

The present disclosure generally relates to a hand-held probe for a Ramen spectroscopy system and a Ramen spectroscopy system.

### BACKGROUND

A plurality of optical techniques has been studied for a wide range of chemical and biomedical applications, owing to their non-invasiveness and rich light-matter interactions. For instance, fluorescence spectroscopy is based on photon absorption and subsequent emission at different energy levels by matter1. Optical coherence tomography utilizes backscattered photons resulting from different reflectivity patterns to obtain the structural information of biological tissues. Visible/infrared spectroscopy relies on the optical absorption, transmission or reflection of matter. Among these techniques, Raman spectroscopy, a measure of photon inelastic scattering, has exceptional specificity over other optical techniques since the resulting spectrum patterns are unique for different compounds. Hence, it has become a popular tool for biomedical applications such as drug design, DNA/RNA analysis and disease diagnosis etc. Confocal Raman spectroscopy (CRS) provides additional spatial resolution in micron level by incorporating a confocal optical setup to suppress stray light and realize depth profiling. Near-infrared laser is often employed as the excitation source for deeper penetration and less autofluorescence. The most commonly used spectrometer in Raman spectroscopy is silicon-based with low noise, high sensitivity and spectral resolution up to sub-nm. Its sensitive region covers 200-1100 nm, corresponding to the fingerprint (FP) region (450 to 1750 cm−1) of most organic chemicals, with 785 nm excitation. To get the high wavenumber (HW) region (2800-3800 cm−1) for water content analysis, several Raman spectroscopy systems adopt dual-wavelength excitation by adding 671 nm laser to broaden the spectral range. However, FP and HW spectra need to be obtained separately during the acquisition by manually switching the laser and repeating the measurement, which may cause depth mismatch and pro-longed acquisition time. This problem is even more pronounced in CRS system operation, especially for in vivo applications.

Therefore, there exists a need for an improved Raman spectroscopy system.

### SUMMARY

According to a first aspect of the present disclosure, a hand-held probe for a Raman spectroscopy system is provided. The hand-held probe may include an excitation module, configured to emit a light beam along a light path, the light path being non-parallel to an observation axis; a beam splitter, arranged in the light path of the light beam emitted by the excitation module and oriented to reflect the light beam along the observation axis; an objective module, disposed along the observation axis and configured to receive the reflected light beam and transmit the reflected light beam to an object to be observed by the objective module so as to generate a scattered light beam by the object, and further configured to receive the scattered light beam from the object and transmit the scattered light beam to the beam splitter so as to generated a refracted light beam by the beam splitter; a focusing mirror, disposed in the observation axis and configured to focus the refracted light beam from the beam splitter to a focus so as to generate a focused light beam; and an emission module, disposed at the focus and configured to receive the focused light beam from the focusing mirror, wherein the emission module is disposed at a same side as the excitation module with respect to the observation axis.

According to a second aspect of the present disclosure, a Raman spectroscopy system is provided. The Raman spectroscopy may include: the hand-held probe as described herein; and a six-axis mechanical arm, wherein the hand-held probe is mounted on the six-axis mechanical arm, wherein the six-axis mechanical arm includes a first joint disposed on a body of the Raman spectroscopy system, a second joint, a third joint disposed on the hand-held probe, a first extension movably connected to the first joint and the second joint, and a second extension movably connected to the second joint and the third joint.

### BRIEF DESCRIPTION OF THE DRAWINGS

FIG. 1 is a block diagram depicting a hand-held probe for an exemplary Raman spectroscopy system according to various embodiments of the present disclosure.

FIG. 2 is a block diagram depicting a hand-held probe for an exemplary Raman spectroscopy system according to various embodiments of the present disclosure.

FIG. 3 is a diagram illustrating a Raman spectroscopy system having a hand-held probe 300 an according to various embodiments of the present disclosure.

FIG. 4 is a diagram illustrating a Raman spectroscopy system having a hand-held probe 400 an according to various embodiments of the present disclosure.

### DETAILED DESCRIPTION

Embodiments described below in the context of a device, apparatus, or system are analogously valid for the respective methods, and vice versa. Furthermore, it will be understood that the embodiments described below may be combined, for example, a part of one embodiment may be combined with a part of another embodiment, and a part of one implementation may be combined with a part of another implementation.

It should be understood that the singular terms “a”, “an”, and “the” include plural references unless context clearly indicates otherwise. Similarly, the word “or” is intended to include “and” unless the context clearly indicates otherwise.

It will be further understood that the terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more steps or elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Approximating language, as used herein throughout the specification and claims, may be applied to modify any quantitative representation that could permissibly vary without resulting in a change in the basic function to which it is related. Accordingly, a value modified by a term or terms, such as “about,” “substantially”, is not limited to the precise value specified but within tolerances that are acceptable for operation of the embodiment for an application for which it is intended. In some instances, the approximating language may correspond to the precision of an instrument for measuring the value.

Various aspects of what is described here seek to provide a hand-held probe for a Raman spectroscopy system. The hand-held probe may be based on a module design, including an excitation module, a beam splitter, an objective module, a focusing mirror (e.g. an optical module with a focusing mirror) and an emission module. The excitation module may be a fibre-coupled input of light, which may consist of two wavelengths (e.g. 671 nm and 785 nm) laser beams. A collimating lens may be used to collimate the laser beam from the single-mode fibre. In some embodiments, a short-pass filter may be disposed between the collimating lens and the beam splitter to pass the beam with wavelength shorter than a cut-off wavelength. In other embodiments, a dual-passband laser cleaning filter (DPLCF) may be disposed between the collimating lens and the beam splitter to eliminate the fluorescence or other unwanted wavelengths from the fibre. The beam splitter (e.g. dichroic beam splitter) may be placed after the DPLCF to reflect the dual-wavelength light beam to the objective module.

While the dual-laser beam shoot on the object, both the Rayleigh scattering and the Raman scattering (e.g. two confocal Raman signals) may be collected by the objective module. A majority (~90%) of Rayleigh scattering may be reflected by the beam splitter to the excitation module, while remaining 10% will be blocked by a long-pass filter with a cut-on wavelength (e.g. 809 nm) disposed between the beam splitter and the focusing mirror. Instead of using a lens, a focusing mirror after the long-pass filter may reflect and focus the emitted Raman scattering into a multi-mode fibre. Advantageously, the emission module can be placed at the same side of the excitation module, which makes the probe more compact and convenient for handheld operation.

According to various aspects, the proposed method may provide an advantageous design of a miniaturized hand-held confocal Raman probe with simultaneous dual-wavelength excitation through a compact wavelength combiner multiplexer. The proposed probe may improve signal collection efficiency by 30% and have a reduced size. By using a short-pass filter, a dichroic beam splitter, and a long-pass filter to replace notch filters in conventional Raman probe, it is possible to cascade two different wavelength laser beams into one multi-mode fibre. This may reduce the system size while improving the power collection efficiency. The proposed fibre-based hand-held design may increase the versatility of the probe for application in different scenario and on different surfaces, like measurements on the face. The dual-wavelength excitation may broaden the Raman spectral range, which may obtain the fingerprint (FP) region together with the high wavenumber (HW) region simultaneously. The FP and HW spectra may be separated from the combined Raman spectra, thus reducing the sampling time by a factor of two.

The following examples pertain to various aspects of the present disclosure.

Example 1 is a hand-held probe for a Raman spectroscopy system, including: an excitation module, configured to emit a light beam along a light path, the light path being non-parallel to an observation axis; a beam splitter, arranged in the light path of the light beam emitted by the excitation module and oriented to reflect the light beam along the observation axis; an objective module, disposed along the observation axis and configured to receive the reflected light beam and transmit the reflected light beam to an object to be observed by the objective module so as to generate a scattered light beam by the object, and further configured to receive the scattered light beam from the object and transmit the scattered light beam to the beam splitter so as to generated a refracted light beam by the beam splitter; a focusing mirror, disposed in the observation axis and configured to focus the refracted light beam from the beam splitter to a focus so as to generate a focused light beam; and an emission module, disposed at the focus and configured to receive the focused light beam from the focusing mirror, wherein the emission module is disposed at a same side as the excitation module with respect to the observation axis.

In Example 2, the subject matter of Example 1 may optionally include a collimating lens, wherein the collimating lens is disposed between the excitation module and the beam splitter and configured to collimate the light beam emitted by the excitation module.

In Example 3, the subject matter of Example 2 may optionally include a short-pass filter having a cut-off wavelength, wherein the short-pass filter is disposed between the collimating lens and the beam splitter in a manner that the light beam emitted by the excitation module passes through the short-pass filter prior to being reflected by the beam splitter.

In Example 4, the subject matter of any Examples 1 to 3 may optionally include a long-pass filter having a cut-on wavelength, wherein the long-pass filter is disposed between the beam splitter and the focusing mirror in a manner that the refracted light beam from the beam splitter passes through the long-pass filter prior to being focused by the focusing mirror.

In Example 5, the subject matter of any Examples 1 to 4 may optionally include that the objective module includes an objective lens assembly, configured to focus the reflected light beam by the beam splitter onto the object and collimate the scattered light beam received from the object.

In Example 6, the subject matter of any Examples 1 to 5 may optionally include that the light beam emitted by the excitation module is a combination of a first laser beam and a second laser beam via a wavelength division multiplexer (WDM).

In Example 7, the subject matter of Example 6 may optionally include that the first laser beam has a wavelength in the range of 630 to 690 nm and the second laser beam has a wavelength in the range of 750 to 830 nm.

In Example 8, the subject matter of any Examples 1 to 7 may optionally include that the light beam emitted by the excitation module is a combination of a first laser beam and a second laser beam via a fibre switch, the fibre switch being configured to switch between the first laser beam and the second laser beam.

In Example 9, the subject matter of Example 4 may optionally include that one part of a Rayleigh scatter portion of the scattered light beam received by the objective module from the object is reflected by the beam splitter and the remaining part of the Rayleigh scatter portion of the scattered light beam is blocked by the long-pass filter.

In Example 10, the subject matter of any Examples 1 to 9 may optionally include a housing having a cylindrical main body and a protruding end extending from the cylindrical main body, wherein the beam splitter, the objective module and the focusing mirror are disposed in the cylindrical main body of the housing.

In Example 11, the subject matter of Example 10 may optionally include that the protruding end of the housing includes an annulus structure having an opening, and a support structure extending from opposing ends of the annulus structure of the protruding end to the cylindrical main body of the housing, wherein a gap exists between the annulus structure and the cylindrical main body of the housing in a manner that the opening of the annulus is accessible from the gap.

In Example 12, the subject matter of Example 11 may optionally include that the opening of the annulus structure of the protruding end is in alignment with the objective module.

In Example 13, the subject matter of Example 11 may optionally include that the annulus structure of the protruding end is configured to sit on a surface of the object.

In Example 14, the subject matter of any Examples 10 to 13 may optionally include that the housing includes a recess disposed on an exterior surface thereof and configured to receive a hand or at least one finger of a user.

In Example 15, the subject matter of any Examples 1 to 14 may optionally include that the excitation module includes a single-mode fibre configured to emit the light beam and disposed along an excitation axis, and wherein the emission module includes a multi-mode fibre configured to receive the focused light beam from the focusing mirror and disposed along a receiving axis.

In Example 16, the subject matter of Example 15 may optionally include that the excitation axis is parallel to the receiving axis.

In Example 17, the subject matter of Example 15 may optionally include that the excitation axis and the receiving axis are perpendicular to the observation axis.

In Example 18, the subject matter of Example 5 may optionally include that the objective module further includes a miniaturized motor stage and the objective lens assembly is mounted on the miniaturized motor stage, wherein the miniaturized motor stage is configured to actuate the objective lens assembly to achieve a confocal measurement.

Example 19 is a Raman spectroscopy system, including: the hand-held probe of Example 1; and a six-axis mechanical arm, wherein the hand-held probe is mounted on the six-axis mechanical arm, wherein the six-axis mechanical arm includes a first joint disposed on a body of the Raman spectroscopy system, a second joint, a third joint disposed on the hand-held probe, a first extension movably connected to the first joint and the second joint, and a second extension movably connected to the second joint and the third joint.

In Example 20, the subject matter of Example 19 may optionally include a spectrometer connected to the emission module, wherein the light beam emitted by the excitation module is a combination of a first laser beam and a second laser beam via a wavelength division multiplexer (WDM), and wherein the spectrometer is configured to record a combined Raman signal measured by the Raman spectroscopy system, the combined Raman signal being a Raman signal obtained from the Raman spectroscopy system when the light beam emitted by the excitation module is the combination of the first laser beam and the second laser beam.

In the following description, example configurations and systems/devices are first described that may employ the techniques described herein. Example details and methods are then described which may be performed in the example configurations and by the systems/devices as well as in other configurations and by other systems/devices. Consequently, implementation of the example details and methods is not limited to the example configurations and systems/devices, and the example configurations and systems/devices are not limited to the example details and methods.

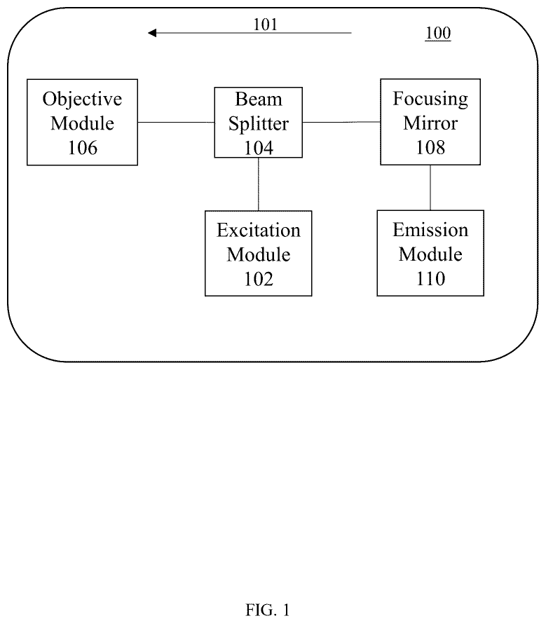

FIG. 1 is a block diagram depicting a hand-held probe 100 for an exemplary Raman spectroscopy system according to various embodiments of the present disclosure. The working principle of Raman spectroscopy may be based upon the interaction of light with the chemical bonds within a material. When a high-intensity laser beam shoots on the surface of a material, a molecule may scatter incident light into two modes: a Rayleigh Scatter that has the same wavelength of the incident light, and a Raman Scatter with different wavelengths depending on the chemical structure of the analyte. The Raman Scattered light may show some peaks that correspond to a specific molecular bond vibration within the Raman spectrum wavelength range. Therefore, a Raman spectroscopy may figure out the chemical structure of a material and provide useful information including the chemical components' identity, intrinsic stress/strain, phase and polymorphism, and contamination and impurity.

According to various non-limiting embodiments, the hand-held probe 100 may include an excitation module 102, a beam splitter 104, an objective module 106, a focusing mirror 108 and an emission module 110. The beam splitter 104 and the focusing mirror 108 may be combined and considered as an optical module. The excitation module 102 may be configured to emit a light beam along a light path, the light path being non-parallel to an observation axis 101. The excitation module 102 may include a single-mode fibre. The excitation module 102 may be further configured to emit dual-wavelength laser beam. For example, the light beam emitted by the excitation module may be a combination of a first laser beam and a second laser beam via a wavelength division multiplexer (WDM). In another example, the light beam emitted by the excitation module may be a combination of a first laser beam and a second laser beam via a fibre switch, the fibre switch being configured to switch between the first laser beam and the second laser beam. The dual-wavelength excitation may broaden the Raman spectral range, which may obtain the fingerprint (FP) region together with the high wavenumber (HW) region simultaneously/quasi-simultaneously. For example, the excitation module 102 may include a fibre-coupled input of light, which consists of two wavelengths (e.g. 671 nm and 785 nm) laser beams. In various embodiments, the first laser beam may have a wavelength in the range of 630 to 690 mm (e.g. 671 nm) and the second laser beam may have a wavelength in the range of 750 to 830 mm (e.g. 785 nm). The difference between the two wavelengths may be in the range of 60 nm to 200 nm, preferably, in the range of 90 nm to 140 nm

According to various non-limiting embodiments, the beam splitter 104 may be arranged in the light path of the light beam emitted by the excitation module 102 and oriented to reflect the light beam along the observation axis 101. A beam splitter (or beamsplitter) is an optical component used for splitting light into two separate beams, usually by wavelength or polarity. The beam splitter 104 may be configured to reflect short-wavelength light beam to the objective module 106. In various embodiments, the beam splitter 104 may include a dichroic beam splitter (or dichroic mirror). The dichroic beam splitter may transmit selected wavelengths while reflecting others. It should be appreciated that the beam splitter 130 may include plate beam splitters, or cube beam splitters in terms of configuration, or polarizing beam splitters or non-polarizing beam splitters in terms of function.

According to various non-limiting embodiments, the objective module 106 may be disposed along the observation axis 101 and configured to receive the reflected light beam from the beam splitter 104 and transmit the reflected light beam to an object (not shown) to be observed by the objective module 106 so as to generate a scattered light beam by the object. The objective module 106 may be further configured to receive the scattered light beam from the object and transmit the scattered light beam to the beam splitter 104 so as to generated a refracted light beam by the beam splitter 104. The scattered light beam from the object may include Rayleigh scatter and Raman scatter beams. A majority (e.g. around 90%) of the Rayleigh scatter beam may be reflected by the beam splitter 104 to the excitation module 102, while a remaining minority (e.g. 10%) of the Rayleigh scatter beam may be transmitted by the beam splitter 104 to the focusing mirror 108. Stated differently, one part, e.g. approximately 90%, of a Rayleigh scatter portion of the scattered light beam received by the objective module 106 from the object may be reflected by the beam splitter 104 and the remaining part, e.g. approximately 10%, of the Rayleigh scatter portion of the scattered light beam may be transmitted by the beam splitter 104.

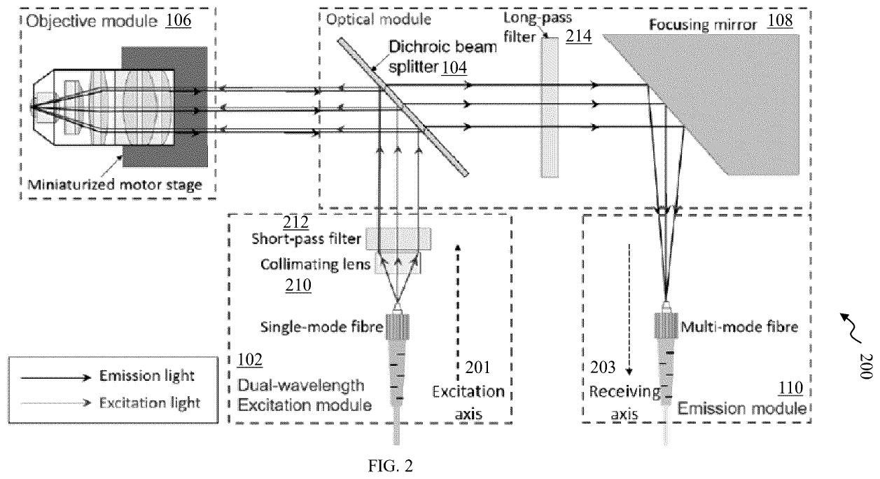

According to various non-limiting embodiments, the objective module 106 may include an objective lens assembly (e.g. see FIG. 2), configured to focus the reflected light beam by the beam splitter 104 onto the object and collimate the scattered light beam received from the object. The objective lens assembly may have high magnification and numerical aperture (NA) for depth profiling. The objective module 106 may be mounted with a (high precision) miniaturized linear motor, which actuates the objective lens assembly to achieve confocal measurement in various depths (e.g. moving along the observation axis 101). Advantageously, synchronization of data collection and depth profiling may be automatically performed by a computing device.

According to various non-limiting embodiments, the focusing mirror 108 may be disposed in the observation axis 101 and configured to focus the refracted light beam from the beam splitter 104 to a focus so as to generate a focused light beam. That may mean the focusing mirror 108 may adjust the transmission direction of the refracted light beam from the beam splitter 104 by a certain angle (e.g. 45° or 90°). The focusing mirror 108 may adjust the transmission direction of the refracted light beam from the beam splitter 104 by reflecting the refracted light beam from the beam splitter 104 at the certain angle.

According to various non-limiting embodiments, the emission module 110 may be disposed at the focus and configured to receive the focused light beam from the focusing mirror 108. In various embodiments, a multi-mode fibre to a near-infrared (NIR) spectrometer (i.e. the emission module) may be disposed at the focus in a manner that the focusing mirror 108 may be configured to focus the refracted light beam from the beam splitter 104 to the multi-mode fibre to the near-infrared (NIR) spectrometer. The emission module 110 may be disposed at a same side as the excitation module 102 with respect to the observation axis 101. Advantageously, the emission module 110 can be placed at the same side of the excitation module 102, which makes the hand-held probe 100 more compact and convenient for hand-held operation.

FIG. 2 is a block diagram depicting a hand-held probe 200 for an exemplary Raman spectroscopy system according to various embodiments of the present disclosure. The hand-held probe 200 may include the features of the hand-held probe 100. In other words, the hand-held probe 200 may include an excitation module 102, a beam splitter 104, an objective module 106, a focusing mirror 108 and an emission module 110. Features that are described in the context of the hand-held probe 100 may correspondingly be applicable to the same or similar features in the hand-held probe 200, and vice versa. Furthermore, additions and/or combinations and/or alternatives as described for a feature in the context of the hand-held probe 100 may correspondingly be applicable to the same or similar feature in the hand-held probe 200, and vice versa.

According to various non-limiting embodiments, the hand-held probe 100 may further include a collimating lens 210. The collimating lens 210 may be disposed between the excitation module 102 and the beam splitter 104 and configured to collimate the light beam emitted by the excitation module 102.

According to various non-limiting embodiments, the hand-held probe 200 may further include a short-pass filter 212 having a cut-off wavelength. The short-pass filter 212 may be disposed between the collimating lens 210 and the beam splitter 104 in a manner that the light beam emitted by the excitation module 102 passes through the short-pass filter prior to being reflected by the beam splitter 104. In other words, the light beam emitted by the excitation module 102 having wavelength longer than the cut-off wavelength of the short-pass filer 212 may not be transmitted by the short-pass filer 212.

According to various non-limiting embodiments, the hand-held probe 200 may include a long-pass filter 214 having a cut-on wavelength. The long-pass filter 214 may be disposed between the beam splitter 104 and the focusing mirror 108 in a manner that the refracted light beam from the beam splitter 104 passes through the long-pass filter 214 prior to being focused by the focusing mirror 108. In other words, the refracted light beam from the beam splitter 104 having wavelength shorter than the cut-off wavelength of the long-pass filer 214 may not be transmitted by the long-pass filer 214. That may mean, one part, e.g. approximately 90%, of a Rayleigh scatter portion of the scattered light beam received by the objective module 106 from the object may be reflected by the beam splitter 104 and the remaining part, e.g. approximately 10%, of the Rayleigh scatter portion of the scattered light beam may be transmitted by the beam splitter 104 and blocked by the long-pass filter 214.

According to various non-limiting embodiments, the excitation module 102 may include a single-mode fibre configured to emit the light beam and disposed along an excitation axis 201. The emission module 110 may include a multi-mode fibre configured to receive the focused light beam from the focusing mirror 108 and disposed along a receiving axis 203. The excitation axis 201 may be parallel to the receiving axis 110. The excitation axis 201 and the receiving axis 203 may be perpendicular to the observation axis. Advantageously, the emission module 110 can be placed at the same side of the excitation module 102, which makes the hand-held probe 200 more compact and convenient for hand-held operation.

According to various non-limiting embodiments, the objective module 106 may further include a miniaturized motor stage and the objective lens assembly may be mounted on the miniaturized motor stage, wherein the miniaturized motor stage is configured to actuate the objective lens assembly to achieve a confocal measurement.

According to various non-limiting embodiments, an exemplary assembling method of the hand-held probe 200 is described herein. The objective module 106 may be firstly assembled with the optical module including the beam splitter 104 and the focusing mirror 108, then the excitation module 102 may be mounted on a five-axis alignment system that may actuate the excitation module 102 moving in X, Y, Z axis, rotating in the horizontal plane and tilting in the vertical plane. An assembler may place an optical profiler after the objective lens, and observer the shape and uniformity of the light spot to adjust the position of the excitation module 102. While the excitation module 102 gets the correct position, it may be fixed by epoxy resin. After the excitation module 102 is fixed at the correct position, the emission module 110 may be aligned with the same method. The emission module 110 may mount on the FAAS and connect to an optical power meter, the position of the emission module 110 may be locked while the power meter gets the maximum reading, and then fixed by epoxy resin.

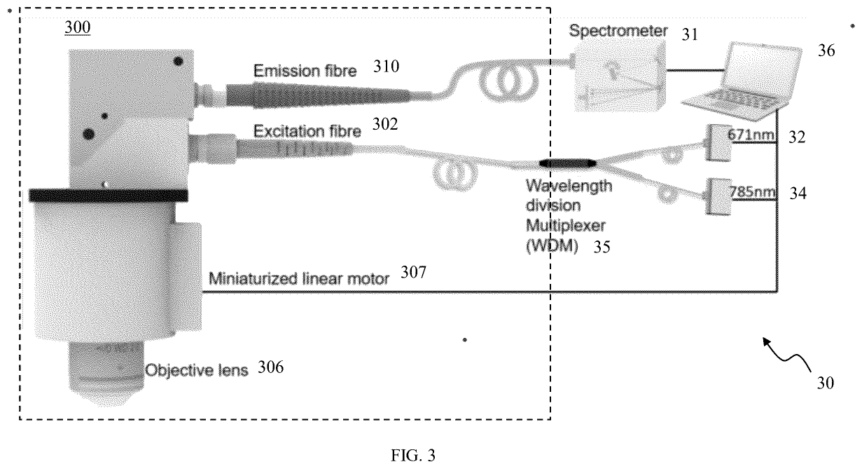

FIG. 3 is a diagram illustrating a Raman spectroscopy system 30 having a hand-held probe 300 an according to various embodiments of the present disclosure. The hand-held probe 300 may include the features of the hand-held probes 100, 200 as described herein with reference to FIGS. 1 and 2. In other words, the hand-held probe 300 may include an excitation module 302 (e.g. an excitation fibre), a beam splitter (not shown), an objective module 306 (e.g. objective lens), a focusing mirror (not shown) and an emission module 310 (e.g. an emission fibre). Features that are described in the context of the hand-held probes 100, 200 may correspondingly be applicable to the same or similar features in the hand-held probe 300, and vice versa. Furthermore, additions and/or combinations and/or alternatives as described for a feature in the context of the hand-held probes 100, 200 may correspondingly be applicable to the same or similar feature in the hand-held probe 300, and vice versa.

According to various non-limiting embodiments, the Raman spectroscopy system 30 may further include a spectrometer 31 (e.g. a near-infrared (NIR) spectrometer), a wavelength division multiplexer (WDM) 35 configured to combine a first laser beam 32 (e.g. having a wavelength 671 nm) and a second laser beam 34 (e.g. having a wavelength 785 nm) and a computing device 36. In some embodiments, the two continuous wave (CW) lasers of 671 and 785 nm may be shot simultaneously through the single-mode fibre (SMF) with 0.39 NA, and combined by the high-transmission-efficiency (>90%), 2-to-1 WDM (NR75A1, Thorlabs) into the excitation single-mode fibre 302. The delivered power of 671 and 785 nm lasers may be 5 and 20 mW, respectively. In some embodiments, a microscope objective lens (NIR Apo 60×1.0 W, Nikon) may be mounted on the miniaturized linear motor 307 to implement confocal measurement. The movement step of the motor 307 may be controlled by the computing device 36 (e.g. a laptop) and the position of the objective lens 306 may be auto-calibrated before each measurement to ensure the accuracy of the confocal depth. In some embodiments, the emission fibre 310 may connect to a near-infrared spectrograph (Kymera 193i) with a back-illuminated CCD camera (iDus 416, Andor Technology). The spectra data may be acquired by the computing device 36 for further processing.

Advantageously, the emission module 310 can be placed at the same side of the excitation module 302, which makes the hand-held probe 300 more compact and convenient for hand-held operation.

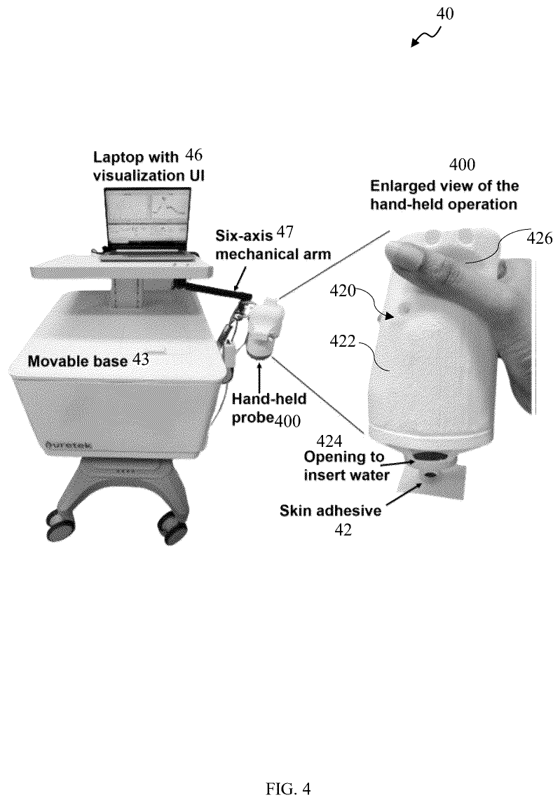

FIG. 4 is a diagram illustrating a Raman spectroscopy system 40 having a hand-held probe 400 an according to various embodiments of the present disclosure. The hand-held probe 400 may include the features of the hand-held probes 100, 200, 300 as described herein with reference to FIGS. 1, 2 and 3. In other words, the hand-held probe 400 may include an excitation module (not shown), a beam splitter (not shown), an objective module (not shown), a focusing mirror (not shown) and an emission module (not shown). Features that are described in the context of the hand-held probes 100, 200, 300 may correspondingly be applicable to the same or similar features in the hand-held probe 400, and vice versa. Furthermore, additions and/or combinations and/or alternatives as described for a feature in the context of the hand-held probes 100, 200, 300 may correspondingly be applicable to the same or similar feature in the hand-held probe 400, and vice versa.

According to various non-limiting embodiments, the hand-held probe 400 may further include a housing 420 having a cylindrical main body 422 and a protruding end 424 extending from the cylindrical main body 422. The beam splitter, the objective module and the focusing mirror may be disposed in the cylindrical main body of the housing 420. The protruding end 424 of the housing 422 may include an annulus structure having an opening, and a support structure extending from opposing ends of the annulus structure of the protruding end 424 to the cylindrical main body 422 of the housing 420. A gap may exist between the annulus structure and the cylindrical main body 422 of the housing 420 in a manner that the opening of the annulus is accessible from the gap. The annulus structure of the protruding end 424 may be configured to sit on a surface of the object. In some embodiments, the opening of the annulus structure of the protruding end 422 may be in alignment with the objective module. In some embodiments, the housing 420 may include a recess 426 disposed on an exterior surface thereof and configured to receive a hand or at least one finger of a user.

According to various non-limiting embodiments, the hand-held probe 400 may be mounted to a six-axis mechanical arm 47, which is attached to a portable base 43, where the rest of the system components may be housed. The arm 47 and base 43 may enable the probe 400 to be placed at any position and angle, thus depth profile data can be acquired from any part of the body. This minimizes movement during measurement as well.

According to various non-limiting embodiments, the objective module of the hand-held probe 400 may include an objective lens of high magnification and numerical aperture (NA), and particularly, a water immersion objective lens may be used to increase the throughput of the system.

According to various non-limiting embodiments, the six-axis mechanical arm 47 may include three joints. The three joints of the mechanical arm 47 may be unlocked, and the protruding end 424 of the probe 400 may be placed on the subject (e.g. on a protruding reservoir of a skin adhesive on the subject). This configuration may enable users to easily place the probe 400 at the desired location without much hassle. Once this is done, the three joints of the mechanical arm 47 may be locked. The objective lens may be then moved closer to the reservoir, such that water surface tension may be maintained. Water may be then filled to the opening 424 of the probe 400 with a syringe and needle. This configuration and workflow may make the probe 400 extremely stable and robust, resulting in fast and good quality imaging and data acquisition.

While this specification contains many details, these should not be understood as limitations on the scope of what may be claimed, but rather as descriptions of features specific to particular examples. Certain features that are described in this specification or shown in the drawings in the context of separate implementations can also be combined. Conversely, various features that are described or shown in the context of a single implementation can also be implemented in multiple implementations separately or in any suitable sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

A number of implementations have been described. Nevertheless, it will be understood that various modifications can be made. Accordingly, other implementations are within the scope of the following claims.

1. A hand-held probe for a Raman spectroscopy system, comprising:
an excitation module, configured to emit a light beam along a light path, the light path being non-parallel to an observation axis;
a beam splitter, arranged in the light path of the light beam emitted by the excitation module and oriented to reflect the light beam along the observation axis;
an objective module, disposed along the observation axis and configured to receive the reflected light beam and transmit the reflected light beam to an object to be observed by the objective module so as to generate a scattered light beam by the object, and further configured to receive the scattered light beam from the object and transmit the scattered light beam to the beam splitter so as to generated a refracted light beam by the beam splitter;
a focusing mirror, disposed in the observation axis and configured to focus the refracted light beam from the beam splitter to a focus so as to generate a focused light beam; and
an emission module, disposed at the focus and configured to receive the focused light beam from the focusing mirror, wherein the emission module is disposed at a same side as the excitation module with respect to the observation axis.

2. The hand-held probe of claim 1, further comprising:
a collimating lens,
wherein the collimating lens is disposed between the excitation module and the beam splitter and configured to collimate the light beam emitted by the excitation module.

3. The hand-held probe of claim 2, further comprising:
a short-pass filter having a cut-off wavelength,
wherein the short-pass filter is disposed between the collimating lens and the beam splitter in a manner that the light beam emitted by the excitation module passes through the short-pass filter prior to being reflected by the beam splitter.

4. The hand-held probe of claim 1, further comprising:
a long-pass filter having a cut-on wavelength,
wherein the long-pass filter is disposed between the beam splitter and the focusing mirror in a manner that the refracted light beam from the beam splitter passes through the long-pass filter prior to being focused by the focusing mirror.

5. The hand-held probe of claim 1, wherein the objective module comprises an objective lens assembly, configured to focus the reflected light beam by the beam splitter onto the object and collimate the scattered light beam received from the object.

6. The hand-held probe of claim 1, wherein the light beam emitted by the excitation module is a combination of a first laser beam and a second laser beam via a wavelength division multiplexer (WDM).

7. The hand-held probe of claim 6, wherein the first laser beam has a wavelength of 671 nm and the second laser beam has a wavelength of 785 nm.

8. The hand-held probe of claim 1, wherein the light beam emitted by the excitation module is a combination of a first laser beam and a second laser beam via a fibre switch, the fibre switch being configured to switch between the first laser beam and the second laser beam.

9. The hand-held probe of claim 4, wherein one part of a Rayleigh scatter portion of the scattered light beam received by the objective module from the object is reflected by the beam splitter and the remaining part of the Rayleigh scatter portion of the scattered light beam is blocked by the long-pass filter.

10. The hand-held probe of claim 1, further comprising:
a housing having a cylindrical main body and a protruding end extending from the cylindrical main body,
wherein the beam splitter, the objective module and the focusing mirror are disposed in the cylindrical main body of the housing.

11. The hand-held probe of claim 10,
wherein the protruding end of the housing comprises an annulus structure having an opening, and a support structure extending from opposing ends of the annulus structure of the protruding end to the cylindrical main body of the housing,
wherein a gap exists between the annulus structure and the cylindrical main body of the housing in a manner that the opening of the annulus is accessible from the gap.

12. The hand-held probe of claim 11, wherein the opening of the annulus structure of the protruding end is in alignment with the objective module.

13. The hand-held probe of claim 11, wherein the annulus structure of the protruding end is configured to sit on a surface of the object.

14. The hand-held probe of claim 10, wherein the housing comprises a recess disposed on an exterior surface thereof and configured to receive a hand or at least one finger of a user.

15. The hand-held probe of claim 1, wherein the excitation module comprises a single-mode fibre configured to emit the light beam and disposed along an excitation axis, and wherein the emission module comprises a multi-mode fibre configured to receive the focused light beam from the focusing mirror and disposed along a receiving axis.

16. The hand-held probe of claim 15, wherein the excitation axis is parallel to the receiving axis.

17. The hand-held probe of claim 15, wherein the excitation axis and the receiving axis are perpendicular to the observation axis.

18. The hand-held probe of claim 5, wherein the objective module further comprises a miniaturized motor stage and the objective lens assembly is mounted on the miniaturized motor stage, wherein the miniaturized motor stage is configured to actuate the objective lens assembly to achieve a confocal measurement.

19. A Raman spectroscopy system, comprising:
the hand-held probe of claim 1; and
a six-axis mechanical arm,
wherein the hand-held probe is mounted on the six-axis mechanical arm,
wherein the six-axis mechanical arm comprises a first joint disposed on a body of the Raman spectroscopy system, a second joint, a third joint disposed on the hand-held probe, a first extension movably connected to the first joint and the second joint, and a second extension movably connected to the second joint and the third joint.

20. The Raman spectroscopy system of claim 19, further comprising:
a spectrometer connected to the emission module,
wherein the light beam emitted by the excitation module is a combination of a first laser beam and a second laser beam via a wavelength division multiplexer (WDM), and
wherein the spectrometer is configured to record a combined Raman signal measured by the Raman spectroscopy system, the combined Raman signal being a Raman signal obtained from the Raman spectroscopy system when the light beam emitted by the excitation module is the combination of the first laser beam and the second laser beam.
